(19) United States
(12) Patent Application Publication
(10) Pub. No.: US 2009/0177138 A1
(43) Pub. Date: Jul. 9, 2009

(54) Shunt Device for Glaucoma Treatment

(76) Inventors: Reay H. Brown, Atlanta, GA (US); Mary Gerard Lynch, Atlanta, GA (US)

Correspondence Address: SUTHERLAND ASBILL & BRENNAN LLP, 999 PEACHTREE STREET, N.E., ATLANTA, GA, 30309, US

(21) Appl. No.: 12/267,032

(22) Filed: Nov. 7, 2008

Related U.S. Application Data
(60) Provisional application No. 60/986,160, filed on Nov. 7, 2007.

(51) Int. Cl. A61M 1/00; A61F 9/007

(52) U.S. Cl. 604/8; 606/108

(57) ABSTRACT

An improved indwelling shunt device for use in treating glaucoma is provided, comprising a flexible plate for placement on the eye, a tube for draining aqueous fluid from the anterior chamber of the eye, said tube having a proximal end and a distal end; and an adjustment element in moveable communication with the proximal region of said tube, adapted to allow modification of the length of the tube extending therefrom. An improved device for engaging the superior and lateral rectus muscles is also provided, comprising a handle; a first hook sized and shaped to engage a rectus muscle; and a second hook sized and shaped to engage a rectus muscle, wherein said second hook is moveable with respect to said first hook.

### CROSS-REFERENCE TO RELATED APPLICATIONS

This application claims priority to U.S. Provisional Patent Application No. 60/986,160 filed on Nov. 7, 2007.

### FIELD OF THE INVENTION

The present invention relates generally to ocular implants and the surgical insertion thereof, and relates more particularly to an implantable shunt device for use in treating glaucoma and a muscle hook device for use during surgical insertion of an ocular implant such as a shunt device.

### BACKGROUND OF THE INVENTION

Glaucoma is a significant public health problem as a major cause of blindness. The blindness that results from glaucoma involves both central and peripheral vision and certainly affects an individual's ability to lead an independent life.

Glaucoma is an optic neuropathy (a disorder of the optic nerve) that usually occurs in the setting of elevated intraocular pressure. The pressure within the eye increases and this is associated with changes in the appearance (“cupping”) and function (“blind spots” in the visual field) of the optic nerve. If the pressure remains high enough for a long period of time, total vision loss occurs. High pressure develops in the eye because of an internal fluid imbalance.

The eye is a hollow structure that contains a clear fluid called “aqueous humor.” Aqueous humor is formed in the posterior chamber of the eye by the ciliary body at a rate of about 2.5 microliters per minute. The fluid, which is made at a fairly constant rate, then passes around the lens, through the pupillary opening in the iris and into the anterior chamber of the eye. Once in the anterior chamber, the fluid drains out of the eye through two different routes. In the “uveoscleral” route, the fluid percolates between muscle fibers of the ciliary body. This route accounts for approximately ten percent of the aqueous outflow in humans. The primary pathway for aqueous outflow in humans is through the “canalicular” route that involves the trabecular meshwork and Schlemm's canal.

The trabecular meshwork and Schlemm's canal are located at the junction between the iris and the sclera. This junction or corner is called “the angle.” The trabecular meshwork is a wedge-shaped structure that runs around the circumference of the eye. It is composed of collagen beams arranged in a three-dimensional sieve-like structure. The beams are lined with a monolayer of cells called trabecular cells. The spaces between the collagen beams are filled with an extracellular substance that is produced by the trabecular cells. The cells also produce enzymes that degrade the extracellular material. Schlemm's canal is adjacent to the trabecular meshwork. The outer wall of the trabecular meshwork coincides with the inner wall of Schlemm's canal. Schlemm's canal is a tube-like structure that runs around the circumference of the cornea.

The aqueous fluid travels through the spaces between the trabecular beams, across the inner wall of Schlemm's canal into the canal, through a series of about 25 collecting channels that drain from Schlemm's canal and into the episcleral venous system. In a normal situation, aqueous production is equal to aqueous outflow and intraocular pressure remains fairly constant in the 15 to 21 mmHg range. In glaucoma, the resistance through the canalicular outflow system is abnormally high.

In primary open angle glaucoma, which is the most common form of glaucoma, the abnormal resistance is believed to be along the outer aspect of trabecular meshwork and the inner wall of Schlemm's canal. It is believed that an abnormal metabolism of the trabecular cells leads to an excessive build up of extracellular materials or a build up of abnormally “stiff” materials in this area. Histopathology of glaucoma eyes also demonstrates a collapse of Schlemm's canal. Primary open angle glaucoma accounts for approximately eighty-five percent of all glaucoma. Other forms of glaucoma (such as angle closure glaucoma and secondary glaucomas) also involve decreased outflow through the canalicular pathway but the increased resistance is from other causes such as mechanical blockage, inflammatory debris, cellular blockage, etc.

With the increased resistance, the aqueous fluid builds up because it cannot exit fast enough. As the fluid builds up, the intraocular pressure (IOP) within the eye increases. The increased IOP compresses the axons in the optic nerve and also may compromise the vascular supply to the optic nerve. The optic nerve carries vision from the eye to the brain. Some optic nerves seem more susceptible to IOP than other eyes. While research is investigating ways to protect the nerve from an elevated pressure, the only therapeutic approach currently available in glaucoma is to reduce the intraocular pressure.

The clinical treatment of glaucoma is approached in a step-wise fashion. Medication often is the first treatment option. Administered either topically or orally, these medications work to either reduce aqueous production or they act to increase outflow. Currently available medications have many serious side effects including: congestive heart failure, respiratory distress, hypertension, depression, renal stones, aplastic anemia, sexual dysfunction and death. Compliance with medication is a major problem, with estimates that over half of glaucoma patients do not follow their correct dosing schedules.

When medication fails to adequately reduce the pressure, laser trabeculoplasty often is performed. In laser trabeculoplasty, thermal energy from a laser is applied to a number of noncontiguous spots in the trabecular meshwork. It is believed that the laser energy stimulates the metabolism of the trabecular cells in some way, and changes the extracellular material in the trabecular meshwork. In approximately eighty percent of patients, aqueous outflow is enhanced and IOP decreases. However, the effect often is not long lasting and fifty percent of patients develop an elevated pressure within five years. The laser surgery is not usually repeatable. In addition, laser trabeculoplasty is not an effective treatment for primary open angle glaucoma in patients less than fifty years of age, nor is it effective for angle closure glaucoma and many secondary glaucomas.

If laser trabeculoplasty does not reduce the pressure enough, then filtering surgery is performed. With filtering surgery, a hole is made in the sclera and angle region. This hole allows the aqueous fluid to leave the eye through an alternate route.

The most commonly performed filtering procedure is a trabeculectomy. In a trabeculectomy, a posterior incision is made in the conjunctiva, the transparent tissue that covers the sclera. The conjunctiva is rolled forward, exposing the sclera at the limbus. A partial thickness scleral flap is made and dissected half-thickness into the cornea. The anterior chamber is entered beneath the scleral flap and a section of deep sclera and trabecular meshwork is excised. The scleral flap is loosely sewn back into place. The conjunctival incision is tightly closed. Post-operatively, the aqueous fluid passes through the hole, beneath the scleral flap and collects in an elevated space beneath the conjunctiva. The fluid then is either absorbed through blood vessels in the conjunctiva or traverses across the conjunctiva into the tear film.

Trabeculectomy is associated with many problems. Fibroblasts that are present in the episclera proliferate and migrate and can scar down the scleral flap. Failure from scarring may occur, particularly in children and young adults. Of eyes that have an initially successful trabeculectomy, eighty percent will fail from scarring within three to five years after surgery. To minimize fibrosis, surgeons now are applying antifibrotic agents such as mitomycin C (MMC) and 5-fluorouracil (5-FU) to the scleral flap at the time of surgery. The use of those agents has increased the success rate of trabeculectomy but also has increased the prevalence of hypotony. Hypotony is a problem that develops when aqueous flows out of the eye too fast. The eye pressure drops too low (usually less than 6.0 mmHg); the structure of the eye collapses and vision decreases.

Trabeculectomy creates a pathway for aqueous fluid to escape to the surface of the eye. At the same time, it creates a pathway for bacteria that normally live on the surface of the eye and eyelids that get into eye. If this happens, an internal eye infection can occur called endophthalmitis. Endophthalmitis often leads to permanent and profound visual loss. Endophthalmitis can occur anytime after trabeculectomy. The risk increases with the thin blebs that develop after MMC and 5-FU. Another factor that contributes to infection is the placement of a bleb. Eyes that have trabeculectomy performed inferiorly have about five times the risk of eye infection than eyes that have a superior bleb. Therefore, initial trabeculectomy is performed superiorly under the eyelid, in either the nasal or temporal quadrant.

In addition to scarring, hypotony and infection, there are other complications of trabeculectomy. The bleb can tear and lead to profound hypotony. The bleb can be irritating and can disrupt the normal tear film, leading to blurred vision. Patients with blebs generally cannot wear contact lenses. All of the complications from trabeculectomy stem from the fact that fluid is being diverted from inside the eye to the external surface of the eye.

When trabeculectomy doesn't successfully lower the eye pressure, the next surgical step is often an aqueous shunt device. There are many aqueous shunt devices in the prior art, which typically involve a drainage tube attached at one end to a plate, as disclosed, for example, in U.S. Pat. No. 4,457,757 to Molteno and U.S. Pat. Nos. 5,558,629 to Baerveldt et.al. To implant an aqueous shunt device, an incision is made in the conjunctiva, exposing the sclera. The plate is sewn to the surface of the eye. A full thickness incision is made into the eye at the limbus, usually with a needle or thin blade. The tube is inserted into the eye through this incision. The external portion of the tube is covered with either donor sclera or pericardium. The conjunctiva is replaced and the incision is closed tightly. An alternative shunt device relying on pressure relief via Schlemm's canal is presented in U.S. Pat. No. 6,464,724.

Many problems are associated with the prior art aqueous shunt devices. One problem involves achieving the correct length for the tube, which must extend from the plate into the anterior chamber of the eye. During the surgical procedure, the tube is typically initially inserted at a length sufficient to extend into the anterior chamber. At the initial length, however, the tube may extend too far into eye, which can cause the patient discomfort and impaired vision. It is often necessary to remove the tube from the eye after its initial placement, trim the tube to a shorter length, and reinsert it. If the tube is still too long, it must be removed and trimmed again, or if it has been cut too short, the tube—and sometimes the entire device—must be replaced. A risk in trimming the tube sequentially is that a single cut may take a tube that is too long and make it too short. Once such a shortening cut has been made, there may be no remedy except replacing the device.

Thus, it is often necessary to remove and adjust or replace the tube multiple times to achieve the desired length. This process is undesirable as it adds time and risk to the surgical procedure. Tube length and design can cause additional problems after the surgical procedure. Even when the tube is implanted at a satisfactory length during the surgical procedure, movement of the eye or plate post-surgery can cause the end of the tube to move with respect to the surface of the anterior chamber. It is not uncommon post-surgery for the end of the tube to slide out of the anterior chamber or for the tube to slide too far into the anterior chamber. Such movement can cause discomfort and impaired vision and can compromise or ruin the functionality of the shunt device. In such cases, further surgery is required to replace the tube.

Another problem associated with the prior art shunt devices is the large incision required for their implantation. A larger incision is associated with a higher risk, as it increases the time required for recovery and carries a higher likelihood of scarring and infection post-surgery.

Even with an incision large enough to fit the plate component of the device, insertion of the plate and suturing of the plate to the eye can be difficult. First, prior art plates are designed to be sutured 8-10 millimeters posterior to the eye limbus—the junction between the sclera and clear cornea. Physician access to this area is difficult without a skilled assistant, and even when access is obtained, suturing is difficult in this area. The difficulty arises because the sclera in this area is particularly thin. The thin sclera increases the risk of penetration of the suture needle and the subsequent creation of a whole in the retina—which lies directly beneath the sclera in this area. This may cause a retinal detachment. Second, depending on the size of the plate, it may extend beneath the superior and lateral rectus muscles, in which case each muscle has to be lifted while one side of the plate is slid beneath the muscle. Prior art muscle hooks for lifting the rectus muscles allow only a small portion of the muscle to be lifted at a time, which does not create a tunnel of sufficient size to fit a full side of the plate.

It would, therefore, be desirable to have an improved aqueous shunt device that overcomes these drawbacks and limitations. In addition, it would be desirable to have an improved muscle hook device for use during surgical insertion of an ocular implant such as a shunt device.

### SUMMARY OF THE INVENTION

Devices for use in the surgical treatment of glaucoma and other eye conditions are provided.

In one aspect, an improved indwelling shunt device for use in treating glaucoma is provided. In one embodiment, the shunt device includes a flexible plate for placement on the eye, a tube for draining aqueous fluid from the anterior chamber of the eye, the tube having a proximal end and a distal end; and an adjustment element in moveable communication with the proximal region of said tube, adapted to allow modification of the length of the tube extending from the plate.

In a different embodiment, the shunt device includes a flexible plate for placement on the eye and a tube for draining aqueous fluid from the anterior chamber of the eye, wherein at least a portion of the tube can be extended or compressed to adjust the length of the tube with respect to the plate.

In another aspect, an improved device for engaging the superior and lateral rectus muscles is provided. The muscle hook device includes a handle; a first hook sized and shaped to engage a rectus muscle; and a second hook sized and shaped to engage a rectus muscle, with the second hook being moveable with respect to the first hook.

### BRIEF DESCRIPTION OF THE DRAWINGS

The objects and advantages of the present invention will be better understood and more readily apparent when considered in conjunction with the following detailed description and accompanying drawings which illustrate, by way of example, preferred embodiments of the shunt device and in which:

FIG. 1 is a schematic view of the eye.

FIG. 2 is a schematic view of one embodiment of the shunt device implanted on an eye.

FIG. 3A is a schematic view showing one embodiment of the tube component of the shunt device. FIG. 3B is a schematic view showing the tube embodiment of FIG. 3A implanted in an eye.

FIG. 4A is a schematic view of one embodiment of the positioning apertures component of the shunt device. FIG. 4B is a schematic view showing another embodiment of the positioning apertures.

FIGS. 5A and 5B are schematic views showing one embodiment of the adjustment element and tube engagement.

FIGS. 6A and 6B are schematic views showing another embodiment of the adjustment element and tube engagement.

FIGS. 7A and 7B are schematic views showing another embodiment of the adjustment element and tube engagement.

FIGS. 8A and 8B are schematic views showing one embodiment of the adjustable tube. FIG. 8A shows the tube in an expanded configuration, and FIG. 8B shows the tube in a compressed configuration.

FIGS. 9A-C are a series of schematic views showing one embodiment of the plate, first in an unfolded configuration in 9A, then in the process of being folded in 9B, then in a folded configuration in 9C.

FIGS. 10A and 10B are schematic views of one embodiment of the muscle hook device.

FIGS. 11A and 11B are schematic views of one embodiment of the muscle hook device.

FIGS. 12A and 12B are cross-sectional views of one embodiment of the muscle hook device in use.

FIG. 13 is a schematic view of one embodiment of the shunt device being implanted on an eye and one embodiment of the muscle hook device being used to lift a rectus muscle.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

An improved shunt device has been developed for use in the surgical treatment of glaucoma. The device comprises a flexible plate and a tube, and advantageously allows adjustment of the length of the tube with respect to the plate.

In one embodiment, the shunt device comprises an adjustment element in moveable communication with the tube, and the adjustment element is adapted to allow adjustment of the tube's length. In one embodiment, the adjustment element is designed to engage with the tube at a plurality of points along the tube. In a further embodiment, the proximal portion of the tube is disposed within the adjustment element. The adjustment element may extend from the distal end of the plate. The adjustment element may comprise positioning apertures for receiving sutures to secure the device to the eye in a location distal to the plate.

In another embodiment of the shunt device, at least a portion of the tube itself can be expanded or contracted to adjust the tube's length. In one embodiment, a portion of the tube may be contracted to reduce the tube's length by pushing the distal and proximal ends of the tube toward one another, and it can be expanded by pulling opposite ends of the tube.

The tube may comprise an anchoring element which extends circumferentially around the distal portion of the tube. The plate may comprise a plurality of scores on at least one of its surfaces, wherein the scores enable the plate to be folded into a pre-determined folded configuration. The width of the plate in the folded configuration may be less than half of the width of the plate when unfolded. The plate may resume an unfolded configuration when unrestrained. in one embodiment, the folded configuration is designed to fit inside an insertion tool, which can be used to insert the plate through a conjunctival incision for implantation. Because the folded configuration reduces the size of the implant upon insertion, it can be passed through a smaller conjunctival incision than an unfolded implant.

In another aspect, an improved muscle hook device has been developed for use in the surgical implantation of an ocular implant. In one embodiment, the hook device comprises a handle, and first and second hooks sized and shaped for engaging a rectus muscle. The second hook is moveable with respect to the first hook.

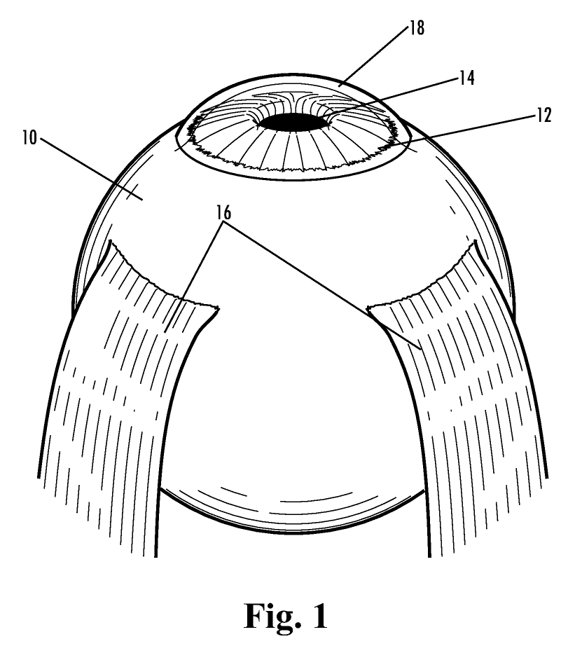

The improved shunt device can be implanted using surgical techniques that are known in the ophthalmological art, such as the procedure described above. FIG. 1 is a simplified view of the relevant anatomy of the eye, and is provided merely for background. FIG. 1 is a schematic view of the eye 10, showing the iris 12, pupil 14, anterior chamber 18, and the superior and lateral rectus muscles 16. Some of the anatomy shown in the figure is not visible during implantation of an ocular implant.

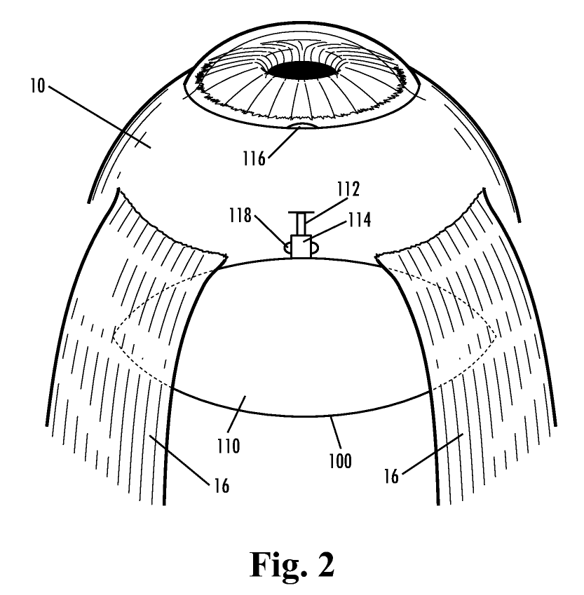

FIG. 2 shows an embodiment of the shunt device 100 implanted in an eye 10, and it more closely approximates the portion of the eye that is visible during the implantation surgery. The device 100 shown comprises a plate 110, a tube 112 with an anchor element 116 at its distal tip, and an adjustment element 114 with positioning apertures 118 extending therefrom. The plate 110, adjustment element 114, and proximal portion of the tube 112 sit atop the sclera. The device 110 is secured to the sclera using sutures sewn through the positioning apertures 118. The sides of the plate 110 are tucked under the rectus muscles 16. The distal portion of the tube 112 extends into the anterior chamber 18 through an incision 20 in the sclera.

The size and shape of the plate 110 can vary. The size and shape should be chosen for implantation in an eye 100. Depending on the size and shape, some of the plate 110 may extend beneath the rectus muscles 16, as shown in FIG. 1, or the entire plate 110 may fit between the rectus muscles 16. The plate 110 shown in FIG. 1 is an oval with pointed sides. The shape of the plate 110 can also be, for example, an ellipse, a circle, or a rectangle with rounded edges. The plate is made of a flexible and biocompatible material, such as silicone, as disclosed in the prior art.

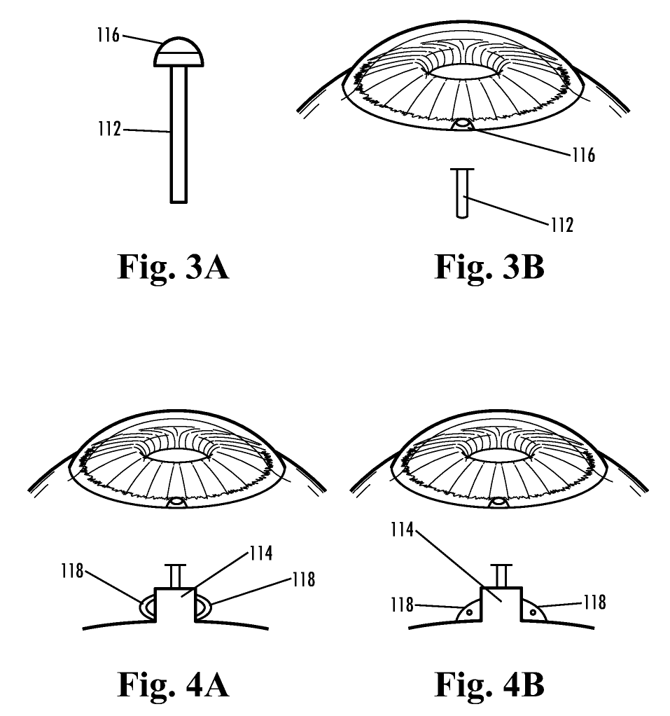

FIG. 3A shows a close up view of one embodiment of the anchor element 116. FIG. 3B shows a close up view of the anchor element 116 embodiment inserted in the anterior chamber 18. The size and shape of the anchor element 116 can vary. The anchor element 116 is preferably only slightly wider than the tube 112, to accommodate inserting the anchor element 116 through an incision 20 in the sclera. Also, the anchor element 116 is preferably wider at its base than at its tip, such as a cone or rounded cone or half-sphere. The anchor element 116 shown in FIG. 3A is a round, approximately spherical cap on the distal end of the tube 112. The anchor element 116 material may be chosen such that the anchor element 116 compresses in width when inserted through the incision 20. If compressed, the anchor element 116 resumes its original width upon insertion.

When implanted, the distal end of the tube 112 should extend into the anterior chamber 18, but preferably reside just inside the anterior chamber 18. When the tube 112 extends too far into the anterior chamber 18, it may cause the patient discomfort and impaired vision. When the distal end of the tube 112 does not reach fully into the anterior chamber 18, the tube 112 cannot perform its intended function of draining aqueous fluid. Thus, it is important for the distal end of the tube 112, once properly inserted, to remain in the anterior chamber 18. The anchor element 116 assists proper placement of the tube 112.

First, the anchor element 116 may be used by the surgeon to detect when the distal end of the tube 112 has reached the anterior chamber 18. Given the size difference, the surgeon will be able to feel when the anchor element 116 is being pressed through the incision 20, as well as when the anchor element 116 has been fully inserted and only the distal anchoring element of the tube 112 extends through the incision 20. Second, the anchor element 116 helps keep the tube 112 in its correct position while it functions. The base of the anchor element 116, which may be round and must be greater than the incision 20 in at least one dimension, rests adjacent to the incision 20 and resists the anchor element 116 sliding backward through the incision 20. This is advantageous, as discussed above, because the tube 112 cannot perform its function of draining aqueous fluid if its distal end is not inside the anterior chamber 18.

Another aspect of the present invention is that the device is sutured to the sclera through holes extending from the adjustment element, rather than through holes in the plate itself. This allows more anterior positioning of the sutures, which is advantageous. More anterior placement of the sutures may allow a smaller incision in the conjunctiva because the location of the sutures is closer to the incision itself. Even with a relatively large incision in the conjunctiva, the surgeon has better access to the holes the closer they are to the anterior chamber. Many prior art plates require suturing the plate in a location on the eye to which the surgeon had poor access, and a skilled assistant is often required to help the surgeon expose the area. Also, more anterior placement of the sutures allows them to be sewn through a thicker layer of sclera. Implantation of many prior art plates requires suturing the plate over the thinnest layer of sclera. Such placement creates a risk of penetration and retinal detachment, which risk is reduced when the sutures are passed through the thicker sclera closer to the anterior chamber.

FIGS. 4A and 4B provide close up views of two embodiments of positioning apertures 118 extending from the adjustment element 114 of the device 100 shown in FIG. 2. The adjustment element 114 extends from the distal end of the plate 110, and the apertures 118 are located distal to the plate 110, such that placement of the apertures 118 by the adjustment element 114 allows more anterior placement of the sutures than would be possible if the sutures were sewn through holes in the plate 110 itself. In FIG. 4A, the apertures 118 are created by round handles extending from the adjustment element 114. In FIG. 4B, the apertures 118 pass through wing-like extensions from the adjustment element 114. These are merely two embodiments of this design feature. The size and shape of the apertures 118 can vary, as can the size and shape of the extensions from the adjustment element 114 that create or surround the apertures 118.

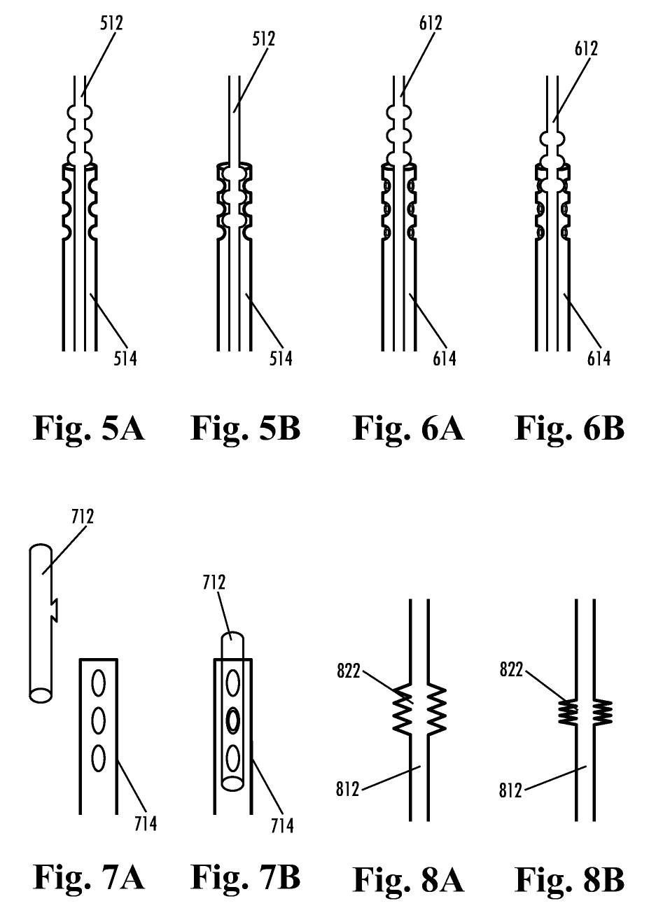

FIGS. 5, 6, and 7 show various embodiments of the adjustment element and tube. In each embodiment, the adjustment element is designed to engage with the tube in a plurality of points along the tube, to fix the tube in place with respect to the adjustment element. FIG. 5 shows one embodiment of the adjustment element 514 and tube 512, in which both the adjustment element and the tube 512 comprise ridges or bumps extending therefrom. The bumps extend from the outer surface of the tube 512, whereas the bumps extend from the inside surface of the adjustment element 514. In FIG. 5A, the portion of the tube 512 comprising such bumps has not yet been inserted into the adjustment element 514, and the bumps of the tube 512 have not yet engaged with the bumps of the adjustment element 514. In FIG. 5B, the tube 512 has been advanced further into the adjustment element 514. The spaces between bumps on the adjustment element 514 are adapted to receive a bump on the tube 512, such that the tube is held in place when the bumps on the tube 512 are positioned between bumps on the adjustment element 514. Although the tube 512 is relatively secure in that position, its location can be intentionally changed by pulling the tube 512 further from the adjustment element 514 or pushing it further into the adjustment element 514, to engage the tube 512 and adjustment element 514 at different points along their length. The length of the tube 512 extending from the adjustment element 514, and correspondingly the length of the tube extending into the anterior chamber, can thereby be adjusted.

In the embodiment shown in FIG. 6, the tube 612 contains ridges or bumps extending from its outer surface, and the adjustment element 614 has holes which extend therethrough and are designed to receive the bumps. In FIG. 6A, the portion of the tube 612 containing bumps has not yet been inserted into the adjustment element 614, so the tube 612 is disengaged from the adjustment element 614. In FIG. 6B, a pair of bumps on the tube 612 is extending into a pair of holes in the adjustment element 614, thereby engaging the tube 612 and adjustment element 614. Again, the position of the tube 612 can be changed by pushing or pulling it with respect to the adjustment element 614. The bumps on the tube 612 will slide through the adjustment element 614 until they enter holes in the adjustment element 614, at which point the tube 612 is movably fixed in that location.

Unlike the embodiments shown in FIGS. 5 and 6, the embodiment shown in FIG. 7 does not involve a portion of the tube being displaced within the adjustment element. Instead, in this embodiment the tube 712 rests atop the adjustment element 714, and one side of the tube 712 engages with one side of the adjustment element 714. FIG. 7A shows a side view of an embodiment of the tube 712 comprising an outer notch, and a front view of an embodiment of the adjustment element 714 comprising holes designed to engage the notch on the tube 712. The notch on the tube 712 can be fit into the holes in the adjustment element 714 to secure the tube to the adjustment element 714. The length of the tube 712 relative to the adjustment element 714 can be modified by moving the notch to a different hole in the adjustment element 714. FIG. 5B shows the tube 712 on top of the adjustment element 714, with the notch engaged in the first hole in the adjustment element 714. The engagement mechanism for the adjustment element and tube is not limited to the embodiments disclosed herein. The engagement can be achieved through any appropriate structure, including bumps, holes, notches, ridges, hinges, and hooks. Also, as described above, an embodiment could be configured with a tubular adjustment element that surrounds the tube. In such an embodiment, holes in the surrounding adjustment element could receive bumps or other structures located on the tube. Alternatively, the adjustment element and tube may be designed to be adjacent during use.

In various embodiments, the tube itself comprises a mechanism for adjustment of its length. One such embodiment is shown in FIG. 8. The embodiment shown comprises an accordian-like region 822, which can be expanded or contracted simply by pushing or pulling the tube 812 on opposite sides of that region 822. FIG. 8A shows the tube 812 in an expanded configuration, and FIG. 8B shows the tube 812 in a contracted configuration. When the tube 812 itself is adjustable, there is no need for an adjustment element in moveable communication with the tube. To achieve advantageous anterior positioning of the sutures, as discussed above, there may be apertures extending from the distal end of the plate, or extending from the tube distal to the plate. Apertures extending from the tube are preferably located proximal to the adjustable portion, such that their location is not affected by expansion or contraction of the tube.

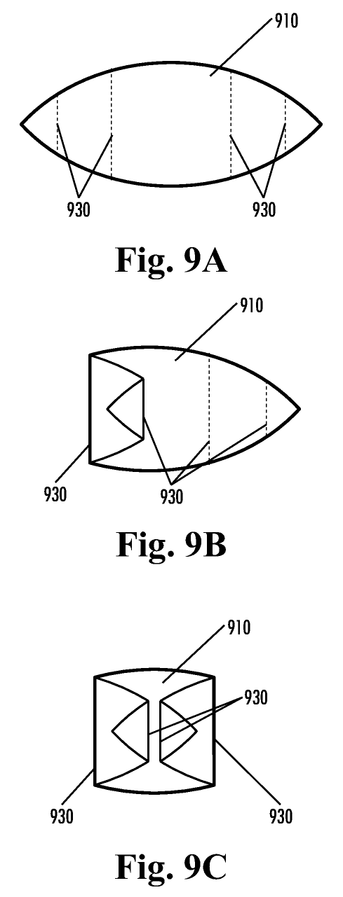

Inserting the plate in a folded configuration may enable a smaller incision in the conjunctiva, as the plate could fit through a smaller incision when folded than when unfolded. The plate may be scored to ease the folding and to accommodate a desirable pre-determined folded configuration. FIGS. 9A-C show one embodiment of a scored plate 910 being folded. The dotted lines represent scoring 930 in a surface of the plate 910. In FIG. 9A, the plate 910 is in an unfolded configuration. In FIG. 9B, one side of the plate 910 has been folded according to the scoring 930, while the other side remains unfolded. In FIG. 9C, the plate 910 is in its predetermined folded configuration. The scoring 930 may be on the top or bottom surface, or both. The scores 930 may be vertical, as shown in FIG. 9, or they may have other configurations, such as horizontal or checkered. The number of scores may also vary. Increasing the number of scores on the plate allows the plate to be folded further and made smaller. The configuration and frequency of the scoring can be chosen to achieve the desired folded configuration of the plate. Vertical scores 930 such as those shown in FIG. 9 allow the width of the plate 910 to be decreased approximately in half. A sufficient number of vertical scores would create a folded configuration in which the plate is approximately rolled.

In its folded configuration, the plate may be placed in an insertion tool, which could take many forms. For example, the insertion tool may have a hollow distal portion designed to hold the folded plate. The loaded insertion tool would be inserted through the conjunctival incision, and the plate would then be pushed from the tool using a plunger-type mechanism. Once expelled from the tool and unrestrained, the plate resumes its unfolded configuration on the eye. The placement of the plate could then be adjusted by the surgeon, such as moving it to the desired distance from the anterior chamber or tucking the sides of the plate beneath the rectus muscles if necessary.

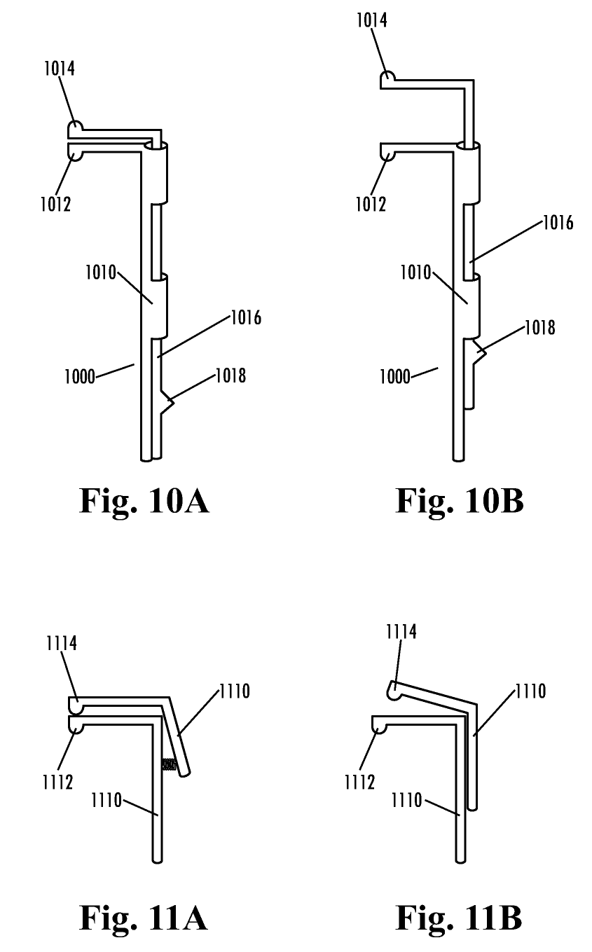

An improved muscle hook device has been developed for use in the surgical implantation of ocular implants. FIGS. 10A and 10B show one embodiment of the muscle hook device, which comprises a handle 1010, and a first hook 1012 and second hook 1014 sized and shaped to engage a rectus muscle. The second hook is moveable with respect to the first hook. In its original configuration, the first hook 1012 and second hooks 1014 are adjacent each other. In FIG. 10B, the second hook 1014 has been slid away from the first hook 1012, separating them. The handle 1010 remains in place while the second hook is moved via an attached sliding lever 1016. The sliding lever 1016 in FIGS. 10A and 10B comprises a grip 1018 that can be used to move the lever 1016.

The hook device embodiment shown in FIG. 11 uses a different mechanism to separate the hooks. The embodiment comprises first and second handles 1110, and a first hook 1112 and second hook 1114. The handles are connected by a spring mechanism, which keeps the handles 1110 separated and the hooks 1112, 1114 together when not in operation. When the handles 1110 are squeezed together, as in FIG. 11B, the hooks 1112, 1114 separate. As shown in FIGS. 10 and 11, the hooks may point in the same direction or in opposite directions. Further, the shape and length of the hooks 1112, 1114 may be adjusted to achieve optimal engagement of the rectus muscles. The length and shape of the handles 1110 may also vary.

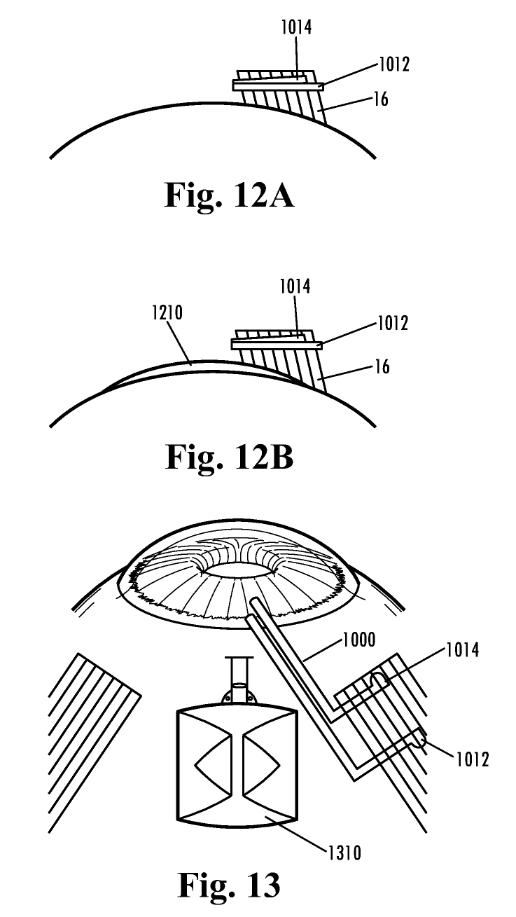

Separation of the hooks, as shown in FIGS. 10B and 11B, is advantageous for lifting a rectus muscle because the two hooks create a larger tunnel beneath the rectus muscle. The muscle hook device 1000 of FIG. 10 is shown in use in the cross-sectional view of FIG. 12. The first hook 1012 and second hook 1014 are separated, such that each hook engages the rectus muscle 16 near its edge, and the hooks are being used to lift the rectus muscle 16 from the sclera. The hooks 1012, 1014 extend beneath the rectus muscle 16, with the hooks parallel to the muscle edge. The handles 1110 are not visible in the cross-sectional view because they extend backward from the hooks 1012, 1014. The muscle 16 passes over the hooks 1012, 1014, creating a tunnel (which is also not visible in the cross-sectional view). In FIG. 12B, a plate 1210 has been slid beneath the rectus muscle 16 while the muscle 16 is lifted.

FIG. 13 also shows the hook device 1000 of FIG. 10 in use. FIG. 13 is a top view of the surgical site with a device inserted with the plate 1310 in a folded configuration and the muscle hook device 1000 being used to lift a rectus muscle 16. The hooks 1012, 1014 are separated to create a tunnel under the rectus muscle 16, at which point one side of the plate 1310 can be unfolded and slide beneath the muscle 16. The procedure is repeated on the other side.

What is claimed is:

1. An indwelling shunt device for use in treating glaucoma, comprising:
a) a flexible plate for placement on the eye, said plate having a top surface, a bottom surface, a proximal end, and a distal end;
b) a tube for draining aqueous fluid from the anterior chamber of the eye, said tube having a proximal end and a distal end; and
c) an adjustment element in moveable communication with the proximal region of said tube, adapted to allow modification of the length of the tube extending therefrom.

2. The device of claim 1, wherein said adjustment element extends from said distal end of said plate.

3. The device of claim 1, wherein said adjustment element is adapted to engage with said tube at a plurality of set points along said tube.

4. The device of claim 1, wherein said adjustment element comprises positioning apertures for receiving a suture.

5. The device of claim 1, wherein the proximal portion of said tube is disposed within said adjustment element.

6. The device of claim 1, wherein said tube comprises an anchor element extending circumferentially around the distal portion of said tube.

7. The device of claim 1, wherein said plate comprises a plurality of scores on at least one of said top and bottom surfaces, said scores being adapted to enable folding of said plate into a pre-determined folded configuration.

8. The device of claim 7, wherein said folded configuration of said plate is adapted to fit within an insertion tool.

9. The device of claim 7, wherein said plate resumes an unfolded configuration when unrestrained.

10. The device of claim 7, wherein the width of said folded configuration is less than half of the width of said plate unfolded.

11. An indwelling shunt device for use in treating glaucoma, comprising:
a) a flexible plate for placement on the eye, said plate having a top surface, a bottom surface, a proximal end, and a distal end; and
b) a tube for draining aqueous fluid from the anterior chamber of the eye, said tube having a proximal end and a distal end, wherein at least a portion of said tube can be extended or compressed to adjust the length of the tube with respect to said distal end of said plate.

12. The device of claim 11, wherein at least a portion of said tube can be compressed by pushing said distal end of said tube toward said proximal end of said tube.

13. The device of claim 11, wherein said tube comprises an anchor element extending circumferentially around the distal portion of said tube.

14. The device of claim 11, wherein said plate comprises a plurality of scores on at least one of said top and bottom surfaces, said scores being adapted to enable folding of said plate into a pre-determined folded configuration.

15. The device of claim 14, wherein said folded configuration of said plate is adapted to fit within an insertion tool.

16. The device of claim 14, wherein said plate resumes an unfolded configuration when unrestrained.

17. The device of claim 14, wherein the width of said folded configuration is less than half of the width of said plate unfolded.

18. A device for engaging the superior and lateral rectus muscles, comprising:
a) A handle;
b) A first hook sized and shaped to engage a rectus muscle;
c) A second hook sized and shaped to engage a rectus muscle, wherein said second hook is moveable with respect to said first hook.
